(19) United States
(12) Patent Application Publication
(10) Pub. No.: US 2016/0346188 A1
(43) Pub. Date: Dec. 1, 2016

(54) Personal Care Cleansing Composition Comprising At Least One Copolymer

(71) Applicants: Prabhjyot Singh, Voorhees, NJ (US); Gundula Starkulla, Mainz (DE); Lisa R. Gandolfi, Fort Mill, SC (US); David H. Johns, York, SC (US)

(72) Inventors: Prabhjyot Singh, Voorhees, NJ (US); Gundula Starkulla, Mainz (DE); Lisa R. Gandolfi, Fort Mill, SC (US); David H. Johns, York, SC (US)

(73) Assignee: Clariant International, Ltd., Muttenz 1 (CH)

(21) Appl. No.: 14/724,453

(22) Filed: May 28, 2015

(51) Int. Cl. A61K 8/81; C08F 222/38; A61Q 5/02; A61Q 19/10; A61K 8/20; A61K 8/49

(52) U.S. Cl. CPC A61K 8/8188; A61K 8/20; A61K 8/4973; A61Q 5/02; A61Q 19/10; C08F 222/38; A61K 2800/48

(57) ABSTRACT

A personal care cleansing composition comprising at least one copolymer X, wherein the copolymer X is synthesized from at least one monomer (A) being acrylamidopropyl-2-methyl-2-sulfonic acid or a salt thereof, at least one monomer (B) conforming to formula (I)

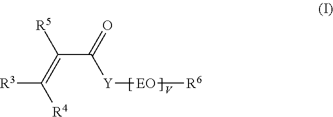

wherein R⁵ is methyl or hydrogen, R³ and R⁴ are hydrogen, Y is oxygen, EO is an ethylene oxide unit, v is an integer from 7 to 25, R⁶ is an alkyl group having from 10 to 22 carbon atoms; and at least one crosslinker (D) conforming to formula (II)

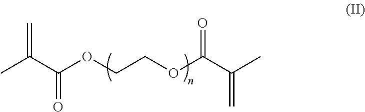

wherein n is an integer from 8 to 15; and wherein the personal care cleansing composition has a pH less than about pH 6.0 and a viscosity of greater than about 1200 cP at 20 rpm.

The present invention relates to personal care cleansing composition comprising at least one copolymer X, wherein the copolymer X synthesized from inter alia at least one monomer (A) being acrylamidopropyl-2-methyl-2-sulfonic acid or a salt thereof and wherein the personal care cleansing composition has a pH less than about pH 6.0, and wherein the personal care cleansing composition has a viscosity of greater than about 1200 cP at 20 rpm.

Cleansing the skin, scalp, and hair is very important for general hygiene and removal of unwanted materials such as sebum, oils, dirt, makeup, and all cosmetic products applied with the intent of beautification. It is a known practice to use foaming cleansing products to clean the skin and hair. It is generally accepted that foaming cleansers need a certain minimum viscosity in order to achieve ease of application to the substrate that will be cleaned. Typically thickening agents are added to surfactants in order to increase the viscosity of the cleansing formulation.

Traditionally the pH of cleanser formulation is adjusted at the end of processing to a pH neutral range, typically between pH 6.5-7.5. Recent advances in preservation technology in personal care formulations have introduced organic acids, such as benzoic acid or sorbic acid, as desirable preservatives for personal care cleansing compositions. Other clinical benefits can be achieved by adding acids such as alpha hydroxy acids or beta hydroxy acids to cleansing formulations, including decreased appearance of wrinkles, skin whitening, and acne reduction. Furthermore, cleansing formulations with a pH of 5.5 have the same pH as the skin and may create a more gentle cleansing personal care cleansing composition for the skin.

With the movement to more acidic personal care cleansing compositions, often below pH 6.0, thickening of the formulation has become a significant challenge and the need has developed for a thickener that provides viscosity increase efficiently at pH below 6.0. It is critical that this thickener also provides all of the cleanser formulation aesthetics that are present at pH 6.5-7.5. Therefore the thickener cannot significantly alter in a negative way the rheological properties, foam behavior, visual appearance, or suspension capabilities of the personal care cleansing composition.

In a first aspect, the present invention relates to a personal care cleansing composition comprising at least one copolymer X, wherein the copolymer X is synthesized from:
a. at least one monomer (A) being acrylamidopropyl-2-methyl-2-sulfonic acid or a salt thereof,
b. at least one monomer (B) conforming to formula (I)

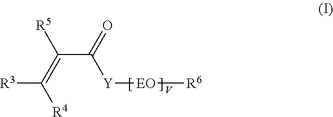

wherein R⁵ is methyl or hydrogen, R³ and R⁴ are hydrogen, Y is oxygen, EO is an ethylene oxide unit, v is an integer from 7 to 25, R⁶ is an alkyl group having from 10 to 22 carbon atoms;
c. optionally at least one further monomer (C), wherein the further monomer (C) is an olefinically unsaturated, non-cationic, optionally crosslinking comonomer which comprises at least one oxygen, nitrogen, sulfur or phosphorus atom and has a molecular weight of less than 500 g/mol; and
d. at least one crosslinker (D) conforming to formula (II)

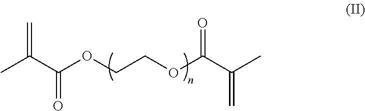

wherein n is an integer from 8 to 15;
   and wherein the personal care cleansing composition has a pH less than about pH 6.0 and a viscosity of greater than about 1200 cP at 20 rpm.

Surprisingly, it has now been found that copolymer X thickens personal care cleansing compositions, such as cosmetic, dermatological, and pharmaceutical cleansing compositions, at pH less than 6.0. Advantageously, the copolymer X also maintains or improves the rheological properties, foam behavior, visual appearance, or suspension capabilities of personal care cleansing compositions even at pH below 6.0.

Thickening of surfactant-based personal care cleansing compositions having a pH of below pH 6.0 can be accomplished in simple fashion by adding electrolytes. Alternatively, cellulose derivatives, natural polymers (e.g., xanthan gum, guar), nonionic surfactants or polyethylene glycol derivatives can be employed. Depending on the method used, however, there are various drawbacks when using such thickening means.

Electrolytes are corrosive; moreover, the thickener effect is heavily dependent on the particular surfactant system (many surfactants cannot be thickened by adding electrolyte). The thickening performance of cellulose derivatives is greatly lowered by salt. Many surfactants, e.g., betaine, contain high quantities of salt, which restricts the use of cellulose derivatives. Natural polymers are very difficult to process; moreover, clear formulations are frequently not realizable. Also, the rheological aesthetics of cleansers thickened by natural polymers is not desired by most consumers. Known nonionic thickeners for liquid surfactant formulations include fatty acid alkanol amides. The fatty acid alkanol amide used preferably in the art is coconut fatty acid diethanol amide. Coconut fatty acid diethanol amide displays the best thickening properties as compared with other fatty acid diethanol amides. A drawback, however, is the presence of amine-type secondary compounds/contaminants. Polyethylene glycol derivatives such as PEG 6000 distearate, PEG-120 methyl glucose dioleate, PEG-150 pentaerythrityl tetrastearate, PEG-20 methyl glucose sesquistearate, etc., are not only complicated to prepare (deficient conformity from batch to batch) but are also in some cases complex to process (melting or dissolution at high temperature, high quantities employed).

Copolymers based on acryloyldimethyltaurine and/or acryloyldimethyltaurates are known where in the art and vary depending on the copolymer structure, degree of crosslinking, and other possible parameters. However, not all copolymers based on acryloyldimethyltaurine and/or acryloyldimethyltaurates are found to provide the same thickening performance in personal care cleansing compositions below pH 6.0.

The thickening ability of the copolymer X in personal care cleansing compositions is intensified by association between the polymer side chains and the surfactants and can be controlled through the choice of the side chains of the polymers and through the choice of surfactants. The copolymer X of the invention also significantly thickens aqueous surfactant systems. The thickening performance is defined as viscosity in centipoise (cP) of the undiluted personal care cleansing composition using an RVDV Brookfield viscometer at 25° C. with 10-90% torque at 20 RPM. The thickening performance is a function of the surfactant system.

The first aspect relates to a personal care cleansing composition. For brevity, the personal care cleansing composition may be referred to as the composition hereinafter.

The composition comprises at least one copolymer X, which is described in more detail below.

In at least one embodiment, the composition is a cosmetic cleansing composition, a dermatological cleansing composition or a pharmaceutical cleansing composition. In at least one embodiment, the composition is a skin cleansing composition or a hair shampoo.

In at least one embodiment, the composition is preferably a rinse-off composition, more preferably shampoos, shower baths, shower gels, foam baths, facial washes, baby washes, baby baths, make-up removing cleansers. Modern rinse-off products frequently have a high proportion of active conditioning substances, which may also be in the form of oily fractions—consequently these compositions may be emulsions.

The composition has a pH less than pH 6.0. Low pH personal care cleansing compositions are useful in view of milder to skin and low pH allows alternative preservation options to paraben and formaldehyde donors preservatives.

In at least one embodiment, the pH is less than pH 5.5, preferably less than pH 5.0. In at least one preferred embodiment, the pH is from pH 2.0 to pH 5.0, more preferably from pH 3.0 to pH 5.0, or from pH 3.5 to pH 4.5.

The composition has a viscosity of greater than 1200 cP at 20 rpm. High viscosity personal care cleansing compositions are useful in view of suspending the beads for skin exfoliation and avoid running of product from hands during use.

Suspending compositions can be obtained. Suspension is defined as viscosity greater than 1000 cP of the undiluted composition using an RVDV Brookfield viscometer at ambient temperature (21-25 C) with 10-90% torque at 5 RPM. Personal care cleansing compositions containing copolymer X can suspend a variety of insoluble materials, including effects pigments, wax beads, polyethylene beads, other insoluble beads, opacifying materials, pearlescent materials, oil droplets, and/or silicone droplets.

Electrolyte-containing systems are known to be very difficult if not impossible to thicken with polyelectrolytes. A synergistic increase in viscosity occurs with the combination of electrolytes, such as sodium chloride, and copolymer X.

The composition comprises at least one copolymer X. Copolymer X has the advantage that it provides an excellent viscosity level for the composition. Indeed, copolymer X is found to provide improved viscosity-increasing performance versus other copolymers available.

Copolymer X is synthesized from at least one monomer (A), at least one monomer (B), optionally at least one further monomer (C), and at least one crosslinker (D).

Monomer (A) is acrylamidopropyl-2-methyl-2-sulfonic acid or a salt thereof. In at least one embodiment the monomer (A) is an organic or inorganic salts of acryloyldimethyltaurine (acrylamidopropyl-2-methyl-2-sulfonic acid).

Preference is given to the Li⁺, Na⁺, K⁺, Mg⁺⁺, Ca⁺⁺, Al⁺⁺⁺ and/or NH₄⁺ salts. Likewise preferred are the monoalkylammonium, dialkylammonium, trialkylammonium and/or tetraalkylammonium salts, in which the alkyl substituents of the amines may independently of one another be (C₁-C₂₂)-alkyl radicals or (C₂-C₁₀)-hydroxyalkyl radicals. Also preferred is the ammonium salt.

The degree of neutralization of the acryloyldimethyltaurine can be between 0 and 100%, with particular preference being given to a degree of neutralization of more than 80%.

Based on the total mass of the copolymers, the amount of acryloyldimethyltaurine and/or acryloyldimethyltaurates is at least 0.1% by weight, preferably from 20 to 99.5% by weight, more preferably from 50 to 98% by weight.

Monomer (B) conforms to formula (I)

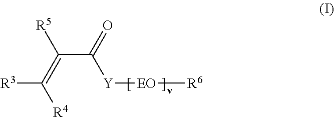

wherein R⁵ is methyl or hydrogen, R³ and R⁴ are hydrogen, Y is oxygen, EO is an ethylene oxide unit, v is an integer from 7 to 25, R⁶ is an alkyl group having from 10 to 22 carbon atoms.

In at least one embodiment, the monomer (B) is an ester of (meth)acrylic acid and a fatty alcohol polyglycol ether selected from the group consisting of: (C₁₀-C₁₈) fatty alcohol polyglycol ethers having 8 EO units, C₁₁ oxo alcohol polyglycol ethers having 8 EO units, (C₁₂-C₁₄) fatty alcohol polyglycol ethers having 7 EO units, (C₁₂-C₁₄) fatty alcohol polyglycol ethers having 11 EO units, (C₁₆-C₁₈) fatty alcohol polyglycol ethers having 8 EO units, (C₁₆-C₁₈) fatty alcohol polyglycol ethers having 15 EO units, (C₁₆-C₁₈) fatty alcohol polyglycol ethers having 11 EO units, (C₁₆-C₁₈) fatty alcohol polyglycol ethers having 20 EO units, (C₁₆-C₁₈) fatty alcohol polyglycol ethers having 25 EO units, (C₁₈-C₂₂) fatty alcohol polyglycol ethers having 25 EO units, iso-(C₁₆-C₁₈) fatty alcohol polyglycol ethers having 25 EO units, and mixtures thereof. In at least one embodiment, the molecular weight of the monomer (B) is preferably from 200 g/mol to 1×10⁶ g/mol, more preferably from 150 g/mol to 1×10⁴ g/mol, even more preferably from 200 to 5000 g/mol.

In at least one embodiment, the copolymer X comprises up to 50.9 wt % monomer (B), by total weight of copolymer X. In at least one embodiment, the copolymer X comprises from 0.5 wt % to 35 wt %, preferably from 5 wt % to 25 wt %, even more preferably 10 wt % to 22.5 wt % monomer (B), by total weight of copolymer X.

In at least one embodiment, the copolymer X is synthesized from at least one further monomer (C). The further monomer (C) is an olefinically unsaturated, non-cationic, optionally crosslinking comonomer which comprises at least one oxygen, nitrogen, sulfur or phosphorus atom and has a molecular weight of less than 500 g/mol. In at least one embodiment, the monomer (C) is not used in the synthesis of copolymer X.

Further preferred monomer (C) are open-chain N-vinyl amides, preferably N-vinylformamide (VIFA), N-vinylmethylformamide, N-vinylmethylacetamide (VIMA) and N-vinylacetamide; cyclic N-vinyl amides (N-vinyl lactams) with a ring size of 3 to 9, preferably N-vinylpyrrolidone (NVP) and N-vinylcaprolactam; amides of acrylic and methacrylic acid, preferably acrylamide, methacrylamide, N,N-dimethyl-acrylamide, N,N-diethylacrylamide, and N,N-diisopropylacrylamide; alkoxylated acrylamides and methacrylamides, preferably hydroxyethyl methacrylate, hydroxymethylmethacrylamide, hydroxyethylmethacrylamide, hydroxypropylmethacrylamide, and mono [2-(methacryloyloxy)ethyl]succinate; N,N-dimethylamino methacrylate; diethylaminomethyl methacrylate; acrylamido- and methacrylamido-glycolic acid; 2- and 4-vinylpyridine; vinyl acetate; glycidyl methacrylate; styrene; acrylonitrile; vinyl chloride; stearyl acrylate; lauryl methacrylate; vinylidene chloride; and/or tetrafluoroethylene.

Likewise suitable monomer (C) are inorganic acids and their salts and esters. Preferred acids are vinylphosphonic acid, vinylsulfonic acid, allylphosphonic acid, and methallylsulfonic acid.

The weight fraction of the monomer (C) based on the total mass of the copolymer X, can be from 0 to 99.8% by weight and is preferably from 0.5 to 80% by weight, more preferably from 2 to 50% by weight.

Copolymer X is synthesized inter alia from crosslinker (D). Crosslinker herein means a comonomer having at least two polymerizable vinyl groups. The crosslinker (D) conforms to formula (II)

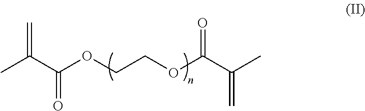

wherein n is an integer from 8 to 15.

The weight fraction of crosslinker (D), based on the total mass of the copolymers, is preferably up to 20% by weight, more preferably from 0.05 to 10% by weight, and very preferably from 0.1 to 7% by weight.

In at least one preferred embodiment, the copolymerization is conducted in the presence of at least one polymeric additive, the additive being added wholly or partly in solution to the polymerization medium before the actual copolymerization. In at least one embodiment, two or more additives is used.

The additives or mixtures thereof must only be wholly or partly soluble in the chosen polymerization medium. During the polymerization step the additive has a number of functions. On the one hand it prevents the formation of overcrosslinked polymer fractions in the copolymer which forms in the polymerization step, and on the other hand the additive is statistically attacked by active free radicals in accordance with the very well-known mechanism of graft copolymerization. Depending on the particular additive, this results in greater or lesser fractions of the additive being incorporated into the copolymers. Moreover, suitable additives possess the property of altering the solution parameters of the copolymers which form during the free-radical polymerization reaction in such a way that the average molecular weights are shifted to higher values. As compared with analogous copolymers prepared without the addition of the additives G), those prepared with the addition of additives advantageously exhibit a significantly higher viscosity in aqueous solution.

Preferred additives are homopolymers and copolymers which are soluble in water and/or alcohols, preferably in t-butanol. The term “copolymers” also comprehends those having more than two different monomer types.

Particularly preferred additives are homopolymers and copolymers of N-vinylformamide, N-vinylacetamide, N-vinylpyrrolidone, ethylene oxide, propylene oxide, acryloyldimethyltaurine, N-vinylcaprolactam, N-vinylmethylacetamide, acrylamide, acrylic acid, methacrylic acid, N-vinylmorpholide, hydroxyethyl methacrylate, diallyldimethylammonium chloride (DADMAC) and/or [2-(methacryloyloxy)ethyl]-trimethylammonium chloride (MAPTAC); polyalkylene glycols and/or alkylpolyglycols.

Particularly preferred additives are polyvinylpyrrolidones (e.g., Luviskol K15®, K20® and K30® from BASF), poly(N-vinylformamides), poly(N-vinylcaprolactams), and copolymers of N-vinylpyrrolidone, N-vinylformamide and/or acrylic acid, which may also have been partly or fully hydrolyzed.

The molecular weight of the additives is preferably from 10² to 10⁷ g/mol, more preferably from 0.5*10⁴ to 10⁶ g/mol.

The amount in which the polymeric additive is used, based on the total mass of the monomers to be polymerized during the copolymerization, is preferably from 0.1 to 90% by weight, more preferably from 1 to 20% by weight, and with particular preference from 1.5 to 10% by weight.

The polymerization medium used may comprise all organic or inorganic solvents which have a very substantially inert behavior with respect to free-radical polymerization reactions and which advantageously allow the formation of medium or high molecular weights. Those used preferably include water; lower alcohols; preferably methanol, ethanol, propanols, iso-, sec- and t-butanol, very preferably t-butanol; hydrocarbons having 1 to 30 carbon atoms, and mixtures of the aforementioned compounds.

The polymerization reaction takes place preferably in the temperature range between 0 and 150° C., more preferably between 10 and 100° C., either at atmospheric pressure or under elevated or reduced pressure. If desired the polymerization may also be performed under an inert gas atmosphere, preferably under nitrogen.

In order to initiate the polymerization it is possible to use high-energy electro-magnetic rays, mechanical energy, or the customary chemical polymerization initiators, such as organic peroxides, e.g., benzoyl peroxide, tert-butyl hydroperoxide, methyl ethyl ketone peroxide, cumene hydroperoxide, dilauroyl peroxide or azo initiators, such as azodiisobutyronitrile (AIBN), for example.

Likewise suitable are inorganic peroxy compounds, such as (NH₄)₂S₂O₈, K₂S₂O₈ or H₂O₂, for example, where appropriate in combination with reducing agents (e.g., sodium hydrogensulfite, ascorbic acid, iron(II) sulfate, etc.) or redox systems comprising as reducing component an aliphatic or aromatic sulfonic acid (e.g., benzenesulfonic acid, toluenesulfonic acid, etc.).

Serving as the polymerization medium may be any solvents which are very substantially inert in respect of free-radical polymerization reactions and which allow the development of high molecular weights. Use is preferably made of water and lower, tertiary alcohols or hydrocarbons having 3 to 30 carbon atoms. In one particularly preferred embodiment t-butanol is used as the reaction medium. Mixtures of two or more representatives of the potential solvents described are of course likewise in accordance with the invention. This also includes emulsions of mutually immiscible solvents (e.g., water/hydrocarbons). In principle, all kinds of reaction regime leading to the polymer structures of the invention are suitable (solution polymerization, emulsion methods, precipitation methods, high-pressure methods, suspension methods, bulk polymerization, gel polymerization, and so on).

Preferred suitability is possessed by precipitation polymerization, particularly preferred suitability by precipitation polymerization in tert-butanol.

Examples of copolymer X include Aristoflex® BLV from Clariant (INCI name: Ammonium Acryloyldimethyltaurate/Beheneth-25 Methacrylate Crosspolymer). Aristoflex® BLV is obtained from the copolymerization of (A) acrylamidopropyl-2-methyl-2-sulfonic acid or a salt thereof (C) an ester of (meth)acrylic acid and a C22 fatty alcohol polyglycol ether having from 7 to 25 ethylene oxide units and (D) a crosslinker conforming to formula (II)

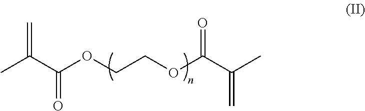

wherein n is an integer of 15.

Clear compositions are useful in view of increased consumer acceptance. Clear compositions can be obtained and are a function of the surfactant system. Clear compositions are defined as those compositions with turbidity less than that are generally considered clear to the naked eye.

The compositions of the invention may as surfactants comprise anionic, nonionic, zwitterionic and/or amphoteric surfactants.

The total amount of the surfactants used, based on the finished composition, is preferably between 2 to 70% by weight, more preferably between 5 and 40% by weight, very preferably between 10 and 35% by weight.

Suitable anionic surfactants include preferably (C₁₀-C₂₀)-alkyl and alkylene carboxylates, alkyl ether carboxylates, fatty alcohol sulfates, fatty alcohol ether sulfates, alkylamide sulfates and sulfonates, fatty acid alkylamide polyglycol ether sulfates, alkanesulfonates and hydroxyalkanesulfonates, olefinsulfonates, acyl esters of isethionates, α-sulfo fatty acid esters, alkylbenzenesulfonates, alkylphenol glycol ether sulfonates, sulfosuccinates, sulfosuccinic monoesters and diesters, fatty alcohol ether phosphates, protein/fatty acid condensation products, alkyl monoglyceride sulfates and sulfonates, alkylglyceride ether sulfonates, fatty acid methyltaurides, fatty acid sarcosinates, sulforicinoleates, and acylglutamates. The compounds and their mixtures are used in the form of their water-soluble or water-dispersible salts, examples being the sodium, potassium, magnesium, ammonium, mono-, di-, and triethanolammonium, and analogous alkylammonium salts.

In at least one embodiment, the composition comprises an acylglycinate surfactant. In at least one embodiment, the acylglycinate surfactant conforms to the formula (Y):

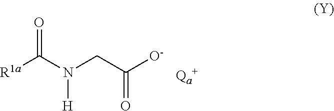

wherein
R^{1a} is a linear or branched, saturated alkanoyl group having 6 to 30, preferably 8 to 22, particularly preferably 8 to 18, carbon atoms or is a linear or branched, mono- or polyunsaturated alkenoyl group having 6 to 30, preferably 8 to 22 and particularly preferably 12 to 18 carbon atoms, and Qₐ⁺ is a cation.

In at least one embodiment, Qₐ⁺ is selected from the group consisting of Li⁺, Na⁺, K⁺, Mg⁺⁺, Ca⁺⁺, Al⁺⁺⁺, NH₄⁺, a monoalkylammmonium ion, a dialkylammonium ion, a trialkylammonium ion and a tetraalkylammonium ion, or combinations thereof. Optionally R^{1a} is independently from one another, are (C₁-C₂₂)-alkyl radicals or (C₂-C₁₀)-hydroxyalkyl radicals. In at least one embodiment, the acylglycinate surfactant is selected from sodium cocoylglycinate and potassium cocoylglycinate. In at least one embodiment, the acylglycinate surfactant is selected from those conforming to formula (Y), wherein R is C12 alkyl or C14 alkyl. In at least one embodiment, the acylglycinate surfactant is selected from those conforming to formula (Y), wherein R is C16 alkyl or C18 alkyl.

In at least one embodiment, the composition comprises a glutamate surfactant corresponding to formula (Z) or a salt thereof:

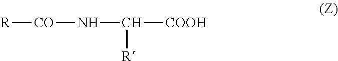

wherein R′ is HOOC—CH₂—CH₂— or M⁺⁻OOC—CH₂—CH₂— wherein M⁺ is a cation; and wherein R is a linear or branched, saturated alkanoyl group having 6 to 30, preferably 8 to 22, more preferably 8 to 18, carbon atoms or is a linear or branched, mono- or polyunsaturated alkenoyl group having 6 to 30, preferably 8 to 22 and more preferably 12 to 18 carbon atoms. In at least one embodiment, M⁺ is a metal cation. In at least one embodiment, M⁺ is selected from the group consisting of Li⁺, Na⁺, K⁺, Mg⁺⁺, Ca⁺⁺, Al⁺⁺⁺, NH₄⁺, a monoalkylammmonium ion, a dialkylammonium ion, a trialkylammonium ion and a tetraalkylammonium ion, or combinations thereof. In at least one embodiment, the glutamate surfactant is selected from sodium cocoyl glutamate and potassium cocoyl glutamate. In at least one embodiment, the glutamate surfactant is selected from those conforming to formula (Z), wherein R is C12 alkyl or C14 alkyl. In at least one embodiment, the glutamate surfactant is selected from those conforming to formula (Z), wherein R is 016 alkyl or C18 alkyl.

The weight fraction of the anionic surfactants, based on the finished composition, is preferably in the range from 2 to 30% by weight, more preferably from 5 to 25% by weight, very preferably from 12 to 22% by weight.

Suitable nonionic surfactants include preferably fatty alcohol ethoxylates (alkylpolyethylene glycols); alkylphenol polyethylene glycols; alkylmercaptan polyethylene glycols; fatty amine ethoxylates (alkylaminopolyethylene glycols); fatty acid ethoxylates (acylpolyethylene glycols); polypropylene glycol ethoxylates (Pluronics®); fatty acid alkylol amides, (fatty acid amide polyethylene glycols); N-alkyl-, N-alkoxypoly-hydroxy-fatty acid amide, sucrose esters; sorbitol esters and polyglycol ethers.

In at least one embodiment, the composition comprises a fatty N-methyl-N-glucosamide surfactant. In at least one embodiment, the fatty N-methyl-N-glucosamide surfactant conforms to the formula (X):

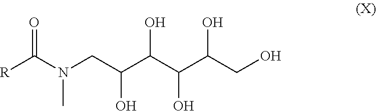

wherein R is a a linear or branched alkyl or alkenyl group having from 3 to 30 carbon atoms. In at least one embodiment, R is an alkyl group having from 3 to 30 carbon atoms. In at least one embodiment, R is a saturated aliphatic hydrocarbon group which can be linear or branched and can have from 3 to 20 carbon atoms in the hydrocarbon chain, preferably linear or branched. Branched means that a lower alkyl group such as methyl, ethyl or propyl is present as substituent on a linear alkyl chain. In at least one embodiment, R is selected from the group consisting of 1-propyl, 2-propyl, 1-butyl, 2-butyl, 2-methyl-1-propyl (isobutyl), 2-methyl-2-propyl (tert-butyl), 1-pentyl, 2-pentyl, 3-pentyl, 2-methyl-1-butyl, 3-methyl-1-butyl, 2-methyl-2-butyl, 3-methyl-2-butyl, 2,2-dimethyl-1-propyl, 1-hexyl, 2-hexyl, 3-hexyl, 2-methyl-1-pentyl, 3-methyl-1-pentyl, 4-methyl-1-pentyl, 2-methyl-2-pentyl, 3-methyl-2-pentyl, 4-methyl-2-pentyl, 2-methyl-3-pentyl, 3-methyl-3-pentyl, 2,2-dimethyl-1-butyl, 2,3-dimethyl-1-butyl, 3,3-dimethyl-1-butyl, 2-ethyl-1-butyl, 2,3-dimethyl-2-butyl, 3,3-dimethyl-2-butyl, 1-heptyl, 1-octyl, 1-nonyl, 1-decyl, 1-undecyl, 1-dodecyl, 1-tetradecyl, 1-hexadecyl and 1-octadecyl. Suitable fatty N-methyl-N-glucosamide surfactants are described in WO2013/178700 and EP 0 550 637, which are incorporated herein by reference. In at least one embodiment, the N-methyl-N-glucosamide surfactant is selected from those conforming to formula (X), wherein R is C12 alkyl or C14 alkyl. In at least one embodiment, the N-methyl-N-glucosamide surfactant is selected from those conforming to formula (X), wherein R is C16 alkyl or C18 alkyl.

The weight fraction of the nonionic surfactants lies preferably in the range from 1 to 20% by weight, more preferably from 2 to 10%, very preferably from 3 to 7% by weight.

Preferred amphoteric surfactants are N—(C₁₂-C₁₈)-alkyl-β-aminopropionates and N—(C₁₂-C₁₈)-alkyl-β-iminodipropionates as alkali metal salts and mono-, di-, and trialkylammonium salts; N-acylaminoalkyl-N,N-dimethylacetobetaine, preferably N—(C₈-C₁₈)-acylaminopropyl-N,N-dimethylacetobetaine; (C₁₂-C₁₈)-alkyl-dimethyl-sulfopropylbetaine; amphosurfactants based on imidazoline (trade name: Miranol®, Steinapon®), preferably the sodium salt of 1-(β-carboxymethyloxyethyl)-1-(carboxy-methyl)-2-laurylimidazolinium; amine oxide, e.g., (C₁₂-C₁₈)-alkyl-dimethylamine oxide, fatty acid amidoalkyldimethylamine oxide.

The weight fraction of the amphoteric surfactants lies preferably in the range from 0.5 to 20% by weight, more preferably from 1 to 10% by weight.

In at least one embodiment, the composition comprises a surfactant system. In at least one embodiment, the surfactant system comprises at least one surfactant selected from the group consisting of lauryl sulfate, laureth sulfate, cocoamidopropylbetaine, sodium cocoylglutamate, lauroamphoacetate, and mixtures thereof. In at least one embodiment, the surfactant system comprises sodium laureth sulphate, sodium lauryl sulphate, and optionally cocamidopropyl betaine. In at least one embodiment, the surfactant system comprises sodium laureth sulphate, Potassium Cocyl Glutamate, and cocamidopropyl betaine. Particularly preferred surfactants are laureth sulfate, cocoamidopropylbetaine, and sodium cocoylglutamate.

As further auxiliaries compositions of the invention may comprise oily substances, emulsifiers, and coemulsifiers, and also further additives common in cosmetology, pharmacy, and dermatology, such as cationic polymers, film formers, superfatting agents, stabilizers, active biogenic substances, glycerol, preservatives, pearlizing agents, dyes and fragrances, solvents, opacifiers, functional acids, and also protein derivatives such as gelatin, collagen hydrolysates, natural or synthetic-based polypeptides, egg yolk, lecithin, lanolin and lanolin derivatives, fatty alcohols, silicones, deodorants, substances with a keratolytic and keratoplastic action, enzymes, and carrier substances. The compositions of the invention may also have antimicrobial agents and preservation boosting ingredients added to them.

An oily substance is any fatty substance which is liquid at room temperature (25° C.).

The fatty phase may therefore comprise one or more oils selected preferably from the following oils: silicone oils, volatile or nonvolatile, linear, branched or cyclic, optionally with organic modification; phenylsilicones; silicone resins and silicone gums; mineral oils such as paraffin oil or vaseline oil; oils of animal origin such as perhydrosqualene, lanolin; oils of plant origin such as liquid triglycerides, e.g., sunflower oil, corn oil, soybean oil, rice oil, jojoba oil, babusscu oil, pumpkin oil, grapeseed oil, sesame oil, walnut oil, apricot oil, macadamia oil, avocado oil, sweet almond oil, lady's-smock oil, castor oil, triglycerides of caprylic/capric acids, olive oil, peanut oil, rapeseed oil, argan oil, abyssinian oil, and coconut oil; synthetic oils such as purcellin oil, isoparaffins, linear and/or branched fatty alcohols and fatty acid esters, preferably guerbet alcohols having 6 to 18, preferably 8 to 10, carbon atoms; esters of linear (C₆-C₁₃) fatty acids with linear (C₆-C₂₀) fatty alcohols; esters of branched (C₆-C₁₃) carboxylic acids with linear (C₆-C₂₆) fatty alcohols, esters of linear (C₆-C₁₈) fatty acids with branched alcohols, especially 2-ethylhexanol; esters of linear and/or branched fatty acids with polyhydric alcohols (such as dimerdiol or trimerdiol, for example) and/or guerbet alcohols; triglycerides based on (C₆-C₁₀) fatty acids; esters such as dioctyl adipate, diisopropyl dimer dilinoleate; propylene glycols/dicaprylate or waxes such as beeswax, paraffin wax or microwaxes, alone or in combination with hydrophilic waxes, such as cetylstearyl alcohol, for example; fluorinated and perfluorinated oils; fluorinated silicone oils; mixtures of the aforementioned compounds.

Suitable nonionogenic coemulsifiers include adducts of from 0 to 30 mol of ethylene oxide and/or from 0 to 5 mol of propylene oxide with linear fatty alcohols having 8 to 22 carbon atoms, with fatty acids having 12 to 22 carbon atoms, with alkylphenols having 8 to 15 carbon atoms in the alkyl group, and with sorbitan or sorbitol esters; (C₁₂-C₁₈) fatty acid monoesters and diesters of adducts of from 0 to 30 mol of ethylene oxide with glycerol; glycerol monoesters and diesters and sorbitan monoesters and diesters of saturated and unsaturated fatty acids having 6 to 22 carbon atoms and, where appropriate, their ethylene oxide adducts; adducts of from 15 to 60 mol of ethylene oxide with castor oil and/or hydrogenated castor oil; polyol esters and especially polyglycerol esters, such as polyglyceryl polyricinoleate and polyglyceryl poly-12-hydroxystearate, for example. Likewise suitable are mixtures of compounds from one or more of these classes of substance.

Examples of suitable ionogenic coemulsifiers include anionic emulsifiers, such as mono-, di- or tri-phosphoric esters, but also cationic emulsifiers such as mono-, di-, and tri-alkyl quats and their polymeric derivatives. Sorbitan caprylate coemulsifier has a synergistic effect on viscosity with copolymer X. The thickening performance is a function of the surfactant system. The weight fraction of sorbitan caprylate is 0.1-3%.

Suitable cationic polymers include those known under the INCI designation Polyquaternium, especially Polyquaternium-31, Polyquaternium-16, Polyquaternium-24, Polyquaternium-7, Polyquaternium-22, Polyquaternium-39, Polyquaternium-28, Polyquaternium-2, Polyquaternium-10, Polyquaternium-11, and also Polyquaternium 37&mineral oil&PPG trideceth (Salcare SC95), PVP-dimethylaminoethyl methacrylate copolymer, guar-hydroxypropyltriammonium chlorides, and also calcium alginate and ammonium alginate. It is additionally possible to employ cationic cellulose derivatives; cationic starch; copolymers of diallylammonium salts and acrylamides; quaternized vinylpyrrolidone/vinylimidazole polymers; condensation products of polyglycols and amines; quaternized collagen polypeptides; quaternized wheat polypeptides; polyethyleneimines; cationic silicone polymers, such as amidomethicones, for example; copolymers of adipic acid and dimethylaminohydroxypropyldiethylenetriamine; polyaminopolyamide and cationic chitin derivatives, such as chitosan, for example.

Examples of suitable silicone compounds are dimethylpolysiloxane, methylphenylpolysiloxanes, cyclic silicones, and amino-, fatty acid-, alcohol-, polyether-, epoxy-, fluoro- and/or alkyl-modified silicone compounds, and also polyalkylsiloxanes, polyalkylarylsiloxanes, polyethersiloxanes, as described in U.S. Pat. No. 5,104,645 and the documents cited therein, which at room temperature may be present either in liquid form or in resin form.

Suitable film formers, depending on the intended application, include salts of phenylbenzimidazolesulfonic acid, water-soluble polyurethanes, for example, C10-polycarbamyl, polyglycerol esters, polyvinyl alcohol, polyvinylpyrrolidone, copolymers thereof, for example vinylpyrrolidone/vinyl acetate copolymer, water-soluble acrylic acid polymers/copolymers and their esters or salts, examples being partial ester copolymers of acrylic/methacrylic acid and polyethylene glycol ethers of fatty alcohols, such as acrylate/steareth-20 methacrylate copolymer, water-soluble cellulose, examples being hydroxymethylcellulose, hydroxyethylcellulose, hydroxypropylcellulose, water-soluble quaterniums, polyquaterniums, carboxyvinyl polymers, such as carbomers and their salts, polysaccharides, polydextrose for example, and glucan.

As superfatting agents it is possible to use substances such as, for example, polyethoxylated lanolin derivatives, lecithin derivatives, polyol fatty acid esters, monoglycerides, and fatty acid alkanol amides, the latter serving simultaneously as foam stabilizers. Moisturizers available include for example isopropyl palmitate, glycerol and/or sorbitol.

As stabilizers it is possible to use metal salts of fatty acids, such as magnesium, aluminum and/or zinc stearate, for example.

Active biogenic substances are to be understood as including, for example, plant extracts and vitamin complexes.

Additionally, the compositions of the invention may comprise organic solvents. Suitable organic solvents include in principle all monohydric or polyhydric alcohols. Preference is given to using alcohols having 1 to 4 carbon atoms such as ethanol, propanol, isopropanol, n-butanol, isobutanol, t-butanol, glycerol, and mixtures of said alcohols. Further preferred alcohols are polyethylene glycols having a relative molecular mass of less than 2000. Particular preference is given to the use of polyethylene glycol having a relative molecular mass of between 200 and 600 in amounts of up to 45% by weight and of polyethylene glycol having a relative molecular mass of between 400 and 600 in amounts of from 5 to 25% by weight. Further suitable solvents are, for example, triacetin (glyceryl triacetate) and 1-methoxy-2-propanol. A hydrotropic action is developed by short-chain anionic surfactants, especially arylsulfonates, for example, cumene sulfonate or toluene sulfonate.

The compositions of the invention can be blended with conventional ceramides, pseudoceramides, fatty acid N-alkylpolyhydroxyalkyl amides, cholesterol, cholesterol fatty acid esters, fatty acids, triglycerides, cerebrosides, phospholipids, and similar substances as a care additive.

Examples of suitable preservatives include benzyl alcohol, piroctone olamine, phenoxyethanol, parabens, pentanediol, benzoic acid/sodium benzoate, sorbic acid/potassium sorbate, and other organic acids used to provide antimicrobial protection. Preservation boosting ingredients include anisic acid, lactic acid, sorbitan caprylate, ethylhexylglycerin, caprylyl glycol, octanediol, and similar substances.

As dyes it is possible to use the substances which are suitable and approved for cosmetic purposes.

Suitable active antifungal substances (fungicides) include preferably ketoconazole, oxiconazole, bifonazole, butoconazole, cloconazole, clotrimazole, econazole, enilconazole, fenticonazole, isoconazole, miconazole, sulconazole, tioconazole, fluconazole, itraconazole, terconazole, naftifine and terbinafine, Zn pyrithione, and octopirox.

Functional acids are acidic substances used to impart a clinical functionality to the skin or hair upon application. Suitable functional acids include alpha hydroxy acids, beta hydroxy acids, lactic acid, retinoic acid, and similar substances.

In a preferred embodiment, the first aspect relates to a personal care cleansing composition comprising at least one copolymer X, wherein the copolymer X is synthesized from:
a. at least one monomer (A) being acrylamidopropyl-2-methyl-2-sulfonic acid or a salt thereof, and
b. at least one monomer (B) conforming to formula (I)

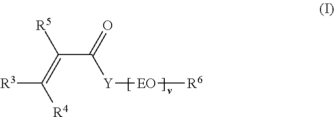

wherein R⁵ is methyl, R³ and R⁴ are hydrogen, Y is oxygen, EO is an ethylene oxide unit, v is an integer from 7 to 25, R⁶ is an alkyl group having from 20 to 22 carbon atoms; and
c. optionally at least one further monomer (C), wherein the further monomer (C) is an olefinically unsaturated, non-cationic, optionally crosslinking comonomer which comprises at least one oxygen, nitrogen, sulfur or phosphorus atom and has a molecular weight of less than 500 g/mol; and
d. at least one crosslinker (D) conforming to formula (II)

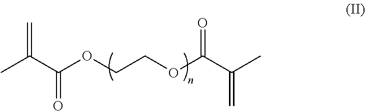

wherein n is an integer from 8 to 15;
   and wherein the composition has a pH less than about pH 5.0,
   and wherein the composition has a viscosity of greater than about 1500 cP at 20 rpm.

In a preferred embodiment, the first aspect relates to a personal care cleansing composition comprising at least one copolymer X, wherein the copolymer X is synthesized from:
a. at least one monomer (A) being acrylamidopropyl-2-methyl-2-sulfonic acid or a salt thereof, and
b. at least one monomer (B) conforming to formula (I)

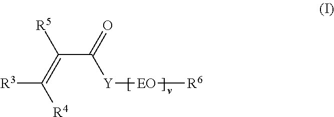

wherein R⁵ is methyl, R³ and R⁴ are hydrogen, Y is oxygen, EO is an ethylene oxide unit, v is an integer from 7 to 25, R⁶ is an alkyl group having from 20 to 22 carbon atoms; and
c. optionally at least one further monomer (C), wherein the further monomer (C) is an olefinically unsaturated, non-cationic, optionally crosslinking comonomer which comprises at least one oxygen, nitrogen, sulfur or phosphorus atom and has a molecular weight of less than 500 g/mol; and
d. at least one crosslinker (D) conforming to formula (II)

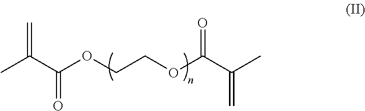

wherein n is an integer from 8 to 15;
   and wherein the composition has a pH less than about pH 5.0;
   and wherein the composition has a viscosity of greater than about 1500 cP at 20 rpm;
   and wherein the composition comprises a surfactant system comprising at least one anionic surfactant;
   and wherein the composition comprises a cosmetically acceptable carrier.

A second aspect relates to a method for cleansing hair and/or skin comprising applying the composition according to the first aspect onto hair and/or skin.

A third aspect relates to a kit comprising the composition according to the first aspect and a formulation selected from the group consisting of hair conditioning formulations, hair styling formulations, intensive conditioning formulations, skin moisturizing formulations, and combinations thereof.

A fourth aspect relates to a container comprising a package comprising a receptacle comprising the composition according to the first aspect and wherein the package comprises a closure for containing the composition in the receptacle.

A fifth aspect relates to the use of the composition according to the first aspect for cleansing hair and/or skin.

The examples which follow are intended to illustrate the subject matter of the invention, though without restricting it thereto. In all compositions, q.s.=“quantum satis”, indicates the specified component is added in the amount needed to obtain the composition goal. q.s. for pH adjusting materials such as citric acid and sodium hydroxide is the amount needed to obtain the desired pH. q.s. for water is the amount needed to obtain a 100% composition

TABLE 1
Example compositions - Copolymer X and
comparative Aristoflex polymers

| | INCI | % Active ingredient (Al) | |
|---|---|---|---|
| A | Polymer | — | 1.25 |
| | Water | q.s | q.s |
| | Surfactant Base I | | |
| B | Sodium Laureth Sulfate (SLES) | 8.00 | 8.00 |
| | Cocamidopropylbetaine (Betaine) | 1.80 | 1.80 |
| | Sodium Cocoyl Glutamate | 1.20 | 1.20 |
| | (Glutamate KCG) | | |
| C | DMDM Hydantoin | 0.4 | 0.4 |
| D | Citric acid | q.s | q.s |

Procedure:
1) Mix components of A until homogenous
2) Mix components of B until homogenous and add to A
3) Add C to 2 and mix until homogeneous
4) Adjust pH with D to desired value
5) Measure viscosity with Brookfield viscometer RVDV-I at 20-23° C.

TABLE 2
Viscosity of compositions with Copolymer X and comparative Aristoflex
polymers

| | | | Polymer | | | |
|---|---|---|---|---|---|---|
| | | | Concentration | | | Viscosity |
| Surfactant base | Polymer | pH | (% Al) | Spindle | RPM | (cP) |
| Surfactant Base I | no polymer | 5.5 | — | 3 | 20 | 11 |
| Surfactant Base I | Aristoflex TAC | 5.5 | 1.25 | 3 | 20 | 385 |
| Surfactant Base I | Aristoflex HMB | 5.5 | 1.25 | 3 | 20 | 360 |
| Surfactant Base I | Aristoflex Velvet | 5.5 | 1.25 | 3 | 20 | 165 |
| Surfactant Base I | COPOLYMER X | 5.5 | 1.25 | 3 | 20 | 4230 |

TABLE 3
Copolymer X Example compositions

| | INCI | % Active ingredient (Al) | | | |
|---|---|---|---|---|---|
| A | Copolymer X | — | 0.75 | 1.0 | 1.25 |
| | Water | q.s | q.s | q.s | q.s |
| | Surfactant Base I | | | | |
| B | Sodium Laureth Sulfate (SLES) | 8.00 | 8.00 | 8.00 | 8.00 |
| | Cocamidopropylbetaine (Betaine) | 1.80 | 1.80 | 1.80 | 1.80 |
| | Sodium Cocoyl Glutamate | 1.20 | 1.20 | 1.20 | 1.20 |
| | (Glutamate KCG) | | | | |
| C | DMDM Hydantoin | 0.4 | 0.4 | 0.4 | 0.4 |
| D | Citric acid | q.s | q.s | q.s | q.s |

1) Mix components of A until homogenous
2) Mix components of B until homogenous and add to A
3) Add C to 2 and mix until homogeneous
4) Adjust pH with D to desired value
5) Measure viscosity with Brookfield viscometer RVDV-I at 20-23° C.

TABLE 4
Viscosity of compositions with Copolymer X

| | | | Polymer | | | |
|---|---|---|---|---|---|---|
| | | | Concentration | | | Viscosity |
| Surfactant base | Polymer | pH | (% Al) | Spindle | RPM | (cP) |
| Surfactant Base I | no polymer | 5.5 | — | 3 | 20 | 11 |
| Surfactant Base I | COPOLYMER X | 5.5 | 1.25 | 3 | 20 | 4230 |
| Surfactant Base I | COPOLYMER X | 5.0 | 1.25 | 3 | 20 | 9600 |
| Surfactant Base I | COPOLYMER X | 4.5 | 1.25 | 3 | 20 | 24000 |
| Surfactant Base I | COPOLYMER X | 5.5 | 1.00 | 3 | 20 | 1435 |
| Surfactant Base I | COPOLYMER X | 5.0 | 1.00 | 3 | 20 | 1775 |
| Surfactant Base I | COPOLYMER X | 4.5 | 1.00 | 3 | 20 | 3775 |
| Surfactant Base I | COPOLYMER X | 4.5 | 0.75 | 3 | 20 | 1965 |

TABLE 5
Example-Copolymer X Example compositions

| | INCI | % Active Ingredient (Al) | | | |
|---|---|---|---|---|---|
| A | Copolymer X | — | 1.00 | — | 1.00 |
| | Water | q.s | q.s | q.s | q.s |
| | | Surfactant Base II | | Surfactant Base III | |
| B | Sodium Laureth Sulfate | 8.00 | 8.00 | 8.00 | 8.00 |
| | AlkylPolyGlucoside | — | — | 2.00 | 2.00 |
| | Lauroyl Methyl Glucamide | 2.00 | 2.00 | — | — |
| C | DMDM Hydantoin | 0.40 | 1.00 | 0.40 | 1.00 |
| D | Citric acid | q.s | q.s | q.s | q.s |

Procedure:
1) Mix components of A until homogenous
2) Mix components of B until homogenous and add to A
3) Add C to 2 and mix until homogeneous
4) Adjust pH with D to desired value
5) Measure viscosity with Brookfield RVDV-I at 20-23° C.

TABLE 6
Viscosity of compositions with Copolymer X

| | | | Polymer | | | |
|---|---|---|---|---|---|---|
| | | | concentration | | | Viscosity |
| Surfactant Base | Polymer | pH | (% Al) | Spindle | RPM | (cP) |
| Surfactant Base II | COPOLYMER X | 5.5 | 0 | 6 | 20 | 0 |
| Surfactant Base II | COPOLYMER X | 5.5 | 1 | 6 | 20 | 1800 |
| Surfactant Base III | COPOLYMER X | 5.5 | 0 | 6 | 20 | 0 |
| Surfactant Base III | COPOLYMER X | 5.5 | 1 | 6 | 20 | 1450 |

TABLE 7
Example-Copolymer X Example compositions

| | INCI | % Active ingredient (Al) | | | |
|---|---|---|---|---|---|
| A | Copolymer X | — | 0.5 | 1.0 | 1.5 |
| | Water | q.s | q.s | q.s | q.s |
| | Surfactant Base IV | | | | |
| B | Genagen 3SB (Coco Betaine, Sodium | 10 | 10 | 10 | 10 |
| | Methyl Cocoyl Taurate, Sodium Cocoyl | | | | |
| | Isethionate) | | | | |
| C | Citric acid | q.s | q.s | q.s | q.s |

Procedure:
1) Mix components of A until homogenous
2) Mix components of B until homogenous and add to A
3) Adjust pH with C to desired value
4) Measure viscosity with Brookfield RVDV-I at 20-23° C.

TABLE 8
Viscosity of compositions with Copolymer X

| | | | Polymer | | | |
|---|---|---|---|---|---|---|
| | | | concentration | Spindle | | Viscosity |
| Surfactant base | Polymer | pH | (% Al) | RV | RPM | (cP) |
| Surfactant Base IV | Copolymer X | 5.5 | — | 2 | 20 | 12 |
| Surfactant Base IV | Copolymer X | 5.5 | 0.5 | 2 | 20 | 156 |
| Surfactant Base IV | Copolymer X | 5.5 | 1.0 | 2 | 20 | 6160 |
| Surfactant Base IV | Copolymer X | 5.5 | 1.5 | 2 | 20 | 13860 |

TABLE 9
Copolymer X Example compositions with organic acid
preservative and sorbitan caprylate

| | INCI | | % Active ingredient | | |
|---|---|---|---|---|---|
| A | Copolymer X | | 1.25 | 1.25 | — |
| | Water | | q.s | q.s | q.s |
| | Surfactant Base I | | | | |
| B | Sodium Laureth Sulfate | | 8.00 | 8.00 | 8.00 |
| | Cocamidopropylbetaine | | 1.80 | 1.80 | 1.80 |
| | Potassium Cocyl Glutamate | | 1.20 | 1.20 | 1.20 |
| C | Sorbitan Caprylate | Clariant | — | 0.67 | 0.67 |
| | Benzoic acid | | — | 0.15 | 0.15 |
| | Propanediol | | — | 0.18 | 0.18 |
| D | Citric acid | Fisher | q.s | q.s | q.s |

Procedure:
1) Mix components of A until homogenous
2) Mix components of B until homogenous and add to A
3) Mix components of C and add to 2. Mix until homogeneous
4) Adjust pH with D to desired value
5) Measure viscosity with Brookfield RVDV-I at 20-23° C.

TABLE 10
Viscosity of compositions with Copolymer X and organic acid preservative and
sorbitan caprylate

| | | Polymer | Organic | | | | |
|---|---|---|---|---|---|---|---|
| | | concentration | acid | | | | Viscosity |
| Surfactant base | Polymer | (% Al) | (% Al) | pH | Spindle | RPM | (cP) |
| Surfactant Base I | COPOLYMER X | 1.25 | — | 5.0 | 6 | 20 | 9600 |
| Surfactant Base I | COPOLYMER X | 1.25 | 0.15 | 5.0 | 6 | 20 | 15400 |
| Surfactant Base I | COPOLYMER X | — | 0.15 | 5.0 | 6 | 20 | 0 |
| Surfactant Base I | COPOLYMER X | 1.25 | — | 5.0 | 6 | 5 | 31000 |
| Surfactant Base I | COPOLYMER X | 1.25 | 0.15 | 5.0 | 6 | 5 | 36600 |
| Surfactant Base I | COPOLYMER X | — | 0.15 | 5.0 | 6 | 5 | 0 |

TABLE 11
Copolymer X Example compositions with sodium chloride

| | INCI | % Active ingredient | | | | |
|---|---|---|---|---|---|---|
| A | Copolymer X | 1.00 | 1.00 | 1.00 | 1.00 | 1.00 |
| | Water | q.s | q.s | q.s | q.s | q.s |
| | Surfactant Base I | | | | | |
| B | Sodium Laureth Sulfate (SLES) | 8.00 | 8.00 | 8.00 | 8.00 | 8.00 |
| | Cocamidopropylbetaine | 1.80 | 1.80 | 1.80 | 1.80 | 1.80 |
| | (Betaine) | | | | | |
| | Sodium Cocyl Glutamate | 1.20 | 1.20 | 1.20 | 1.20 | 1.20 |
| | (Glutamate KCG) | | | | | |
| C | DMDM Hydantoin | 1.00 | 1.00 | 1.00 | 1.00 | 1.00 |
| D | Sodium Chloride (NaCl) | 0.25 | 0.5 | 1.0 | 1.5 | 2.0 |

Procedure:
1) Mix components of A until homogenous
2) Mix components of B until homogenous and add to A
3) Add C to 2 and mix until homogeneous
4) Adjust pH with D to pH 5.0.
5) Measure viscosity with Brookfield RVDV-I at 20-23° C.

TABLE 12
Viscosity of compositions with Copolymer X and sodium chloride

| | | | Polymer | Salt | | | |
|---|---|---|---|---|---|---|---|
| | | | concentration | concentration | | | Viscosity |
| Surfactant Base | Polymer | pH | (% Al) | (% Al) | Spindle | RPM | (cP) |
| Surfactant Base I | no polymer | 5.0 | — | 0 | 6 | 20 | 11 |
| Surfactant Base I | COPOLYMER X | 5.0 | 1.0 | 0 | 6 | 20 | 2200 |
| Surfactant Base I | COPOLYMER X | 5.0 | 1.0 | 0.25 | 6 | 20 | 3100 |
| Surfactant Base I | COPOLYMER X | 5.0 | 1.0 | 0.5 | 6 | 20 | 4200 |
| Surfactant Base I | COPOLYMER X | 5.0 | 1.0 | 1.0 | 6 | 20 | 18250 |
| Surfactant Base I | COPOLYMER X | 5.0 | 1.0 | 1.5 | 6 | 20 | 36000 |
| Surfactant Base I | COPOLYMER X | 5.0 | 1.0 | 2.0 | 6 | 20 | 43400 |

TABLE 13
Copolymer X Example compositions with sodium chloride

| | INCI | | |
|---|---|---|---|
| A | Copolymer X | 0.75 | 1.0 |
| | Water | q.s. | q.s. |
| B | Sodium Laureth Sulfate | 14 | 14 |
| | (SLES) | | |
| C | Sodium Chloride (NaCl) | 0-2.0 | 0-2.0 |
| D | Citric Acid/NaOH | q.s. | q.s. |

Procedure:
1) Mix components of A until homogenous
2) Mix components of B until homogenous and add to A
3) Add C to 2 and mix until homogenous.
4) Adjust pH to 5.5-6.0 with D.
5) Measure viscosity with Brookfield viscometer RVDV-I at 20-23° C.

TABLE 14
Viscosity of compositions with Copolymer X and sodium chloride

| | | | Polymer | Salt | | | |
|---|---|---|---|---|---|---|---|
| Surfactant | | | concentration | concentration | | | Viscosity |
| base | Polymer | pH | (% Al) | (% Al) | Spindle | RPM | (cP) |
| 14% SLES | COPOLYMER X | 5.5-6.0 | 0.75 | — | 2 | 20 | 258 |
| 14% SLES | COPOLYMER X | 5.5-6.0 | 0.75 | 0.5 | 2 | 20 | 220 |
| 14% SLES | COPOLYMER X | 5.5-6.0 | 0.75 | 1.0 | 2 | 20 | 266 |
| 14% SLES | COPOLYMER X | 5.5-6.0 | 0.75 | 2.0 | 2 | 20 | 3000 |
| 14% SLES | COPOLYMER X | 5.5-6.0 | 1.0 | — | 2 | 20 | 772 |
| 14% SLES | COPOLYMER X | 5.5-6.0 | 1.0 | 0.5 | 2 | 20 | 700 |
| 14% SLES | COPOLYMER X | 5.5-6.0 | 1.0 | 1.0 | 2 | 20 | 1140 |
| 14% SLES | COPOLYMER X | 5.5-6.0 | 1.0 | 2.0 | 2 | 20 | 8720 |

TABLE 15
Copolymer X Example compositions with sodium chloride

| | INCI | | | |
|---|---|---|---|---|
| A | Copolymer X | — | 0.75 | 1.0 |
| | Water | q.s. | q.s. | q.s. |
| | Surfactant Base VI | | | |
| B | Sodium Laureth Sulfate | 14 | 14 | 14 |
| | Cocamidopropyl betaine | 1.5 | 1.5 | 1.5 |
| C | Sodium Chloride (NaCl) | 0-2.0 | 0-2.0 | 0-2.0 |
| D | Citric Acid/NaOH | q.s. | q.s. | q.s. |

Procedure:
1) Mix components of A until homogenous
2) Mix components of B until homogenous and add to A
3) Add C to 2 and mix until homogenous.
4) Adjust pH to 5.5-6.0 with D.
5) Measure viscosity with Brookfield viscometer RVDV-I at 20-23° C.

TABLE 16
Viscosity of compositions with Copolymer X and sodium chloride

| | | | Polymer | Salt | | | |
|---|---|---|---|---|---|---|---|
| | | | concentration | concentration | | | Viscosity |
| Surfactant Base | | pH | (% Al) | (% Al) | Spindle | RPM | (cP) |
| Surfactant Base VI | Copolymer X | 5.5-6.0 | — | — | 2 | 20 | 0 |
| Surfactant Base VI | Copolymer X | 5.5-6.0 | — | 0.5 | 2 | 20 | 8 |
| Surfactant Base VI | Copolymer X | 5.5-6.0 | — | 1.0 | 2 | 20 | 16 |
| Surfactant Base VI | Copolymer X | 5.5-6.0 | — | 1.5 | 2 | 20 | 38 |
| Surfactant Base VI | Copolymer X | 5.5-6.0 | — | 2.0 | 2 | 20 | 1050 |
| Surfactant Base VI | Copolymer X | 5.5-6.0 | 0.75 | — | 2 | 20 | 248 |
| Surfactant Base VI | Copolymer X | 5.5-6.0 | 0.75 | 0.5 | 2 | 20 | 522 |
| Surfactant Base VI | Copolymer X | 5.5-6.0 | 0.75 | 1.0 | 2 | 20 | 2485 |
| Surfactant Base VI | Copolymer X | 5.5-6.0 | 0.75 | 1.5 | 2 | 20 | 24200 |
| Surfactant Base VI | Copolymer X | 5.5-6.0 | 0.75 | 2.0 | 2 | 20 | 45400 |
| Surfactant Base VI | Copolymer X | 5.5-6.0 | 1.0 | — | 2 | 20 | 1194 |
| Surfactant Base VI | Copolymer X | 5.5-6.0 | 1.0 | 0.5 | 2 | 20 | 2310 |
| Surfactant Base VI | Copolymer X | 5.5-6.0 | 1.0 | 1.0 | 2 | 20 | 18450 |
| Surfactant Base VI | Copolymer X | 5.5-6.0 | 1.0 | 1.5 | 2 | 20 | 65800 |
| Surfactant Base VI | Copolymer X | 5.5-6.0 | 1.0 | 2.0 | 2 | 20 | 65200 |

TABLE 17
Copolymer X Example compositions and turbidity (clarity)

| | | | Polymer | |
|---|---|---|---|---|
| | | | concentration | Turbidity |
| Surfactant base | Polymer | pH | (% Al) | (NTU) |
| Surfactant Base I | no polymer | 5.5 | — | 3 |
| Surfactant Base I | Aristoflex TAC | 5.5 | 1.25 | 36 |
| Surfactant Base I | Aristoflex HMB | 5.5 | 1.25 | 16 |
| Surfactant Base I | Copolymer X | 5.5 | 1.25 | 44 |
| Surfactant Base I | Aristoflex Velvet | 5.5 | 1.25 | 75 |
| Surfactant Base I | Copolymer X | 5.5 | 1.0 | 18 |
| Surfactant Base I | Copolymer X | 5.0 | 1.0 | 13 |

TABLE 18
Copolymer X Example compositions and suspension capability based on
viscosity at 5 RPM

| | | | Polymer | | | | |
|---|---|---|---|---|---|---|---|
| | | | concentration | | | Viscosity | Suspension |
| Surfactant base | Polymer | pH | (% Al) | Spindle | RPM | (cP) | Score |
| Surfactant Base I | Copolymer X | 5.5 | 1.00 | 6 | 5.0 | 5000 | 4.0 |
| Surfactant Base I | Copolymer X | 5.0 | 1.00 | 6 | 5.0 | 7600 | 4.0 |
| Surfactant Base I | Copolymer X | 4.5 | 1.00 | 6 | 5.0 | 13400 | 4.0 |
| Surfactant Base I | Copolymer X | 5.5 | 1.25 | 6 | 5.0 | 18400 | 4.0 |
| Surfactant Base I | Copolymer X | 5.0 | 1.25 | 6 | 5.0 | 31000 | 3.5 |
| Surfactant Base I | Copolymer X | 4.5 | 1.25 | 6 | 5.0 | 53000 | 3.0 |
| Surfactant Base I | Copolymer X | 5.0 | 0.75 | 6 | 5.0 | 2600 | 2.5 |
| Surfactant Base I | Copolymer X | 4.5 | 0.75 | 6 | 5.0 | 6200 | 2.0 |

TABLE 19
Copolymer X Example compositions and viscosity stability at
temperatures >35 C. and 20 RPM

| | | Polymer | | | Storage | Viscosity (cP) | |
|---|---|---|---|---|---|---|---|
| Surfactant | | concentration | | | temperature | @20 RPM | |
| base | Polymer | (% Al) | Spindle | pH | (C.) | Initial | 4 weeks |
| Surfactant | Copolymer X | 1.00 | 6 | 5.5 | 37 | 1650 | 1900 |
| Base I | | | | | | | |
| Surfactant | Copolymer X | 1.00 | 6 | 5.0 | 37 | 2200 | 2400 |
| Base I | | | | | | | |
| Surfactant | Copolymer X | 1.00 | 6 | 4.5 | 37 | 4250 | 5150 |
| Base I | | | | | | | |
| Surfactant | Copolymer X | 1.25 | 6 | 5.5 | 37 | 5350 | 5700 |
| Base I | | | | | | | |
| Surfactant | Copolymer X | 0.75 | 6 | 5.0 | 37 | 800 | 1000 |
| Base I | | | | | | | |
| Surfactant | Copolymer X | 0.75 | 6 | 4.5 | 37 | 2300 | 3800 |
| Base I | | | | | | | |
| Surfactant | Copolymer X | 1.00 | 6 | 5.5 | 50 | 1650 | 1900 |
| Base I | | | | | | | |
| Surfactant | Copolymer X | 1.00 | 6 | 5.0 | 50 | 2200 | 3150 |
| Base I | | | | | | | |
| Surfactant | Copolymer X | 1.00 | 6 | 4.5 | 50 | 4250 | 7700 |
| Base I | | | | | | | |
| Surfactant | Copolymer X | 1.25 | 6 | 5.5 | 50 | 5350 | 5400 |
| Base I | | | | | | | |
| Surfactant | Copolymer X | 0.75 | 6 | 5.0 | 50 | 800 | 1150 |
| Base I | | | | | | | |
| Surfactant | Copolymer X | 0.75 | 6 | 4.5 | 50 | 2300 | 4700 |
| Base I | | | | | | | |

TABLE 20
Copolymer X Example compositions and viscosity stability at
temperatures >35 C. and 5 RPM

| | | Polymer | | | Storage | Viscosity (cP) | |
|---|---|---|---|---|---|---|---|
| Surfactant | | concentration | | | temperature | @5 rpm | |
| base | Polymer | (% Al) | Spindle | pH | (C.) | Initial | 4 weeks. |
| Surfactant | Copolymer X | 1.00 | 6 | 5.5 | 37 | 5000 | 6000 |
| Base I | | | | | | | |
| Surfactant | Copolymer X | 1.00 | 6 | 5.0 | 37 | 7600 | 8000 |
| Base I | | | | | | | |
| Surfactant | Copolymer X | 1.00 | 6 | 4.5 | 37 | 13400 | 16000 |
| Base I | | | | | | | |
| Surfactant | Copolymer X | 1.25 | 6 | 5.5 | 37 | 18400 | 18800 |
| Base I | | | | | | | |
| Surfactant | Copolymer X | 0.75 | 6 | 5.0 | 37 | 2600 | 3200 |
| Base I | | | | | | | |
| Surfactant | Copolymer X | 0.75 | 6 | 4.5 | 37 | 6200 | 9800 |
| Base I | | | | | | | |
| Surfactant | Copolymer X | 1.00 | 6 | 5.5 | 50 | 5000 | 6000 |
| Base I | | | | | | | |
| Surfactant | Copolymer X | 1.00 | 6 | 5.0 | 50 | 7600 | 9400 |
| Base I | | | | | | | |
| Surfactant | Copolymer X | 1.00 | 6 | 4.5 | 50 | 13400 | 21000 |
| Base I | | | | | | | |
| Surfactant | Copolymer X | 1.25 | 6 | 5.5 | 50 | 18400 | 18400 |
| Base I | | | | | | | |
| Surfactant | Copolymer X | 0.75 | 6 | 5.0 | 50 | 2600 | 3600 |
| Base I | | | | | | | |

What is claimed is:

1. A personal care cleansing composition comprising at least one copolymer X, wherein the copolymer X is synthesized from:
a. at least one monomer (A) being acrylamidopropyl-2-methyl-2-sulfonic acid or a salt thereof,
b. at least one monomer (B) conforming to formula (I) 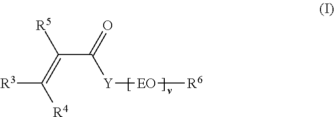
wherein R⁵ is methyl or hydrogen, R³ and R⁴ are hydrogen, Y is oxygen, EO is an ethylene oxide unit, v is an integer from 7 to 25, R⁶ is an alkyl group having from 10 to 22 carbon atoms;
c. optionally at least one further monomer (C), wherein the further monomer (C) is an olefinically unsaturated, non-cationic, optionally crosslinking comonomer which comprises at least one oxygen, nitrogen, sulfur or phosphorus atom and has a molecular weight of less than 500 g/mol; and
d. at least one crosslinker (D) conforming to formula (II) 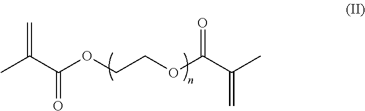
wherein n is an integer from 8 to 15;
and wherein the personal care cleansing composition has a pH less than about pH 6.0 and a viscosity of greater than about 1200 cP at 20 rpm.

2. The personal care cleansing composition according to claim 1, wherein the pH is less than about pH 5.5.

3. The personal care cleansing composition according to claim 1, wherein the composition comprises from about 0.6 wt % to about 1.5 wt % copolymer X, by total weight of the composition.

4. The personal care cleansing composition according to claim 1, further comprising at least one anionic surfactant.

5. The personal care cleansing composition according to claim 1, further comprising at least one amphoteric surfactant.

6. The personal care cleansing composition according to claim 1, further comprising at least one non-ionic surfactant.

7. The personal care cleansing composition according to claim 1, further comprising at least one organic acid preservative.

8. The personal care cleansing composition according to claim 1, further comprising at least one functional acid.

9. The personal care cleansing composition according to claim 1, further comprising sodium chloride.

10. The personal care cleansing composition according to claim 4, further comprising sodium chloride.

11. The personal care cleansing composition according to claim 5, further comprising sodium chloride.

12. The personal care cleansing composition according to claim 6, further comprising sodium chloride.

13. The personal care cleansing composition according to claim 7, further comprising sodium chloride.

14. The personal care cleansing composition according to claim 8, further comprising sodium chloride.

15. The personal care cleansing composition according to claim 1, further comprising 0.1 to 3 wt. % sorbitan caprylate.

16. The personal care cleansing composition according to claim 4, further comprising 0.1 to 3 wt. % sorbitan caprylate.

17. The personal care cleansing composition according to claim 5, further comprising 0.1 to 3 wt. % sorbitan caprylate.

18. The personal care cleansing composition according to claim 6, further comprising 0.1 to 3 wt. % sorbitan caprylate.

19. The personal care cleansing composition according to claim 7, further comprising 0.1 to 3 wt. % sorbitan caprylate.

20. The personal care cleansing composition according to claim 8, further comprising 0.1 to 3 wt. % sorbitan caprylate.

21. The personal care cleansing composition according to claim 1, wherein the pH is less than 5.0.

22. The personal care cleansing composition according to claim 1, wherein the viscosity of the personal care cleansing composition after storing for 4 weeks at ≧35° C. is equal to or greater than 1200 cP at 20 rpm.

23. A personal care cleansing composition comprising at least one copolymer X, wherein the copolymer X is synthesized from:
a. at least one monomer (A) being acrylamidopropyl-2-methyl-2-sulfonic acid or a salt thereof,
b. at least one monomer (B) conforming to formula (I) 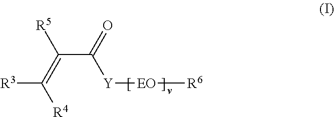
wherein R⁵ is methyl or hydrogen, R³ and R⁴ are hydrogen, Y is oxygen, EO is an ethylene oxide unit, v is an integer from 7 to 25, R⁶ is an alkyl group having from 10 to 22 carbon atoms;
c. optionally at least one further monomer (C), wherein the further monomer (C) is an olefinically unsaturated, non-cationic, optionally crosslinking comonomer which comprises at least one oxygen, nitrogen, sulfur or phosphorus atom and has a molecular weight of less than 500 g/mol; and
d. at least one crosslinker (D) conforming to formula (II) 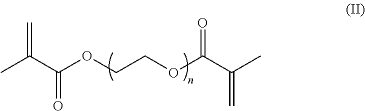
wherein n is an integer from 8 to 15;
and wherein the personal care cleansing composition has a pH less than about pH 6.0 and a viscosity of greater than about 5000 cP at 5 rpm.

24. The personal care cleansing composition according to claim 23, wherein the pH is less than 5.5.

25. The personal care cleansing composition according to claim 23, wherein the concentration of the copolymer X is 0.8-1.5% based on the total weight of the personal care cleanser.

26. The personal care cleansing composition according to claim 23, further comprising at least one anionic surfactant.

27. The personal care cleansing composition according to claim 23, further comprising at least one amphoteric surfactant.

28. The personal care cleansing composition according to claim 23, further comprising at least one non-ionic surfactant.

29. The personal care cleansing composition according to claim 23, further comprising at least one organic acid preservative.

30. The personal care cleansing composition according to claim 23, further comprising at least one functional acid.

31. The personal care cleansing composition according to claim 23, wherein the viscosity of the personal care cleansing composition after storing for 4 weeks at ≧35° C. is equal to or greater than 5000 cP at 5 rpm.
